(19) United States
(12) Patent Application Publication
(10) Pub. No.: US 2024/0016919 A1
(43) Pub. Date: Jan. 18, 2024

(54) INTRADERMAL MERS-CoV VACCINE

(71) Applicant: THE WISTAR INSTITUTE OF ANATOMY AND BIOLOGY, Philadelphia, PA (US)

(72) Inventors: Ami Patel, Philadelphia, PA (US); David Weiner, Merion, PA (US); Kar Muthumani, Philadelphia, PA (US); Emma Reuschel, Philadelphia, PA (US)

(21) Appl. No.: 18/042,796

(22) PCT Filed: Aug. 24, 2021
(86) PCT No.: PCT/US21/47300

§ 371 Date: Feb. 24, 2023

(51) Int. Cl. A61K 39/215; C12N 15/85; C07K 14/165

(52) U.S. Cl. CPC A61K 39/215; C12N 15/85; C07K 14/165; C12N 2770/20022; A61K 2039/5258

(57) ABSTRACT

The present invention disclosed an intradermal vaccine that protects against Middle East Respiratory Syndrome coronavirus (MERS-CoV). In one embodiment, the vaccine is a DNA vaccine. In one embodiment, the vaccine comprises an antigen. The antigen can be a consensus antigen. The consensus antigen can be a consensus spike antigen. The present invention also discloses methods of treating or preventing MERS-CoV in a subject in need thereof by administering the vaccine intradermally to the subject.

### CROSS-REFERENCE TO RELATED APPLICATIONS

This application claims priority to U.S. Provisional Patent Application No. 63/069,868, filed Aug. 25, 202, the contents of which are incorporated by reference herein in its entirety.

### REFERENCE TO A “SEQUENCE LISTING,” A TABLE, OR A COMPUTER PROGRAM LISTING APPENDIX SUBMITTED AS AN ASCII TEXT FILE

The present application hereby incorporates by reference the entire contents of the text file named “206193-0073-00WO_Sequence_Listing_ST25.txt” in ASCII format. The text file containing the Sequence Listing of the present application was created on Aug. 23, 2021 and is 35,232 bytes in size.

### TECHNICAL FIELD OF THE INVENTION

The present invention relates to an intradermal vaccine for Middle East Respiratory Syndrome coronavirus (MERS-CoV) and a method of intradermal delivery of the vaccine.

### BACKGROUND OF THE INVENTION

Middle East respiratory syndrome (MERS) coronavirus (MERS-CoV) is a positive-sense, single-stranded RNA coronavirus that infects the lower and upper respiratory tract, causing a viral pneumonia characterized by acute respiratory symptoms, fever, aches, shortness of breath, sore throat, cough, and diarrhea and vomiting (Guery B et al., 2013, Lancet, 381:2265-2272). Since 2012, there have been 2566 laboratory confirmed cases and 882 MERS-CoV associated deaths (34.4% case fatality rate)(Wu J T et al., 2020, Lancet, 395:689-697). Human cases are frequently associated with close-contact of infected camels, however human-to-human transmission has been observed, nosocomial transmission has been observed, and travel-associated cases have become a global health priority concern.

The 2015 South Korean outbreak originated from a single traveler who returned home from the Middle East. In total, 186 people were infected in the South Korean outbreak, with 36 MERS-associated fatalities (Ki M, 2015, Epidemiol Health, 37:e2015033), resulting in significant impact on the hospital systems of South Korea. This outbreak highlights the importance of rapid infection control for emerging coronaviruses and other infectious diseases. The urgent need for accelerated vaccine development has become critical in light of the ongoing severe acute respiratory syndrome coronavirus 2 (SARS-CoV-2) pandemic, a betacoronavirus related to MERS-CoV.

Thus, there is a need remains in the art for the development of a safe and effective vaccine that is applicable to MERS-CoV, thereby providing protection against and promoting survival of MERS-CoV infection. The present disclosure addresses this unmet need in the art.

### SUMMARY OF THE INVENTION

The present invention is directed to an intradermal vaccine comprising an immunogenic composition. In one embodiment, the present invention is directed to an intradermal vaccine comprising a nucleic acid molecule, wherein the nucleic acid molecule can comprise a nucleic acid sequence having at least about 90% identity over an entire length of the nucleic acid sequence set forth in SEQ ID NO:1 or the nucleic acid molecule can comprise a nucleic acid sequence having at least about 90% identity over an entire length of the nucleic acid sequence set forth in SEQ ID NO:3.

In one embodiment, the nucleic acid molecule comprises a nucleic acid sequence set forth in SEQ ID NO:1.

In one embodiment, the nucleic acid molecule comprises a nucleic acid sequence set forth in SEQ ID NO:3.

The present invention is also directed to an intradermal vaccine comprising a nucleic acid molecule, wherein the nucleic acid molecule can encode a peptide comprising an amino acid sequence having at least about 90% identity over an entire length of the amino acid sequence set forth in SEQ ID NO:2 or the nucleic acid molecule can encode a peptide comprising an amino acid sequence having at least about 90% identity over an entire length of the amino acid sequence set forth in SEQ ID NO:4.

In one embodiment, the nucleic acid molecule encodes a peptide comprising an amino acid sequence set forth in SEQ ID NO:2.

In one embodiment, the nucleic acid molecule encodes a peptide comprising an amino acid sequence set forth in SEQ ID NO:4.

The present invention is further directed to an intradermal vaccine comprising an antigen, wherein the antigen is encoded by SEQ ID NO:1 or SEQ ID NO:3.

The present invention is also directed to an intradermal vaccine comprising a peptide, wherein the peptide can comprise an amino acid sequence having at least about 90% identity over an entire length of the amino acid sequence set forth in SEQ ID NO:2 or the peptide can comprise an amino acid sequence having at least about 90% identity over an entire length of the amino acid sequence set forth in SEQ ID NO:4.

The present invention is further directed to a method of inducing an immune response against a Middle East Respiratory Syndrome coronavirus (MERS-CoV) in a subject in need thereof. The method can comprise administering one or more of the above vaccines intradermally to the subject.

The present invention is further directed to a method of protecting a subject in need thereof from infection with a MERS-CoV. The method can comprise administering one or more of the above vaccines intradermally to the subject.

The present invention is further directed to a method of treating a subject in need thereof against MERS-CoV. The method can comprise administering one or more of the above vaccines intradermally to the subject.

### BRIEF DESCRIPTION OF THE DRAWINGS

FIG. 1, comprising FIG. 1A through FIG. 1E, depicts representative study timeline and immune responses induced by MERS-CoV DNA vaccine. FIG. 1A depicts a representative immunization and blood collection timeline. Rhesus macaques (n=6) were immunized intramuscularly (IM) with 1 mg or intradermally (ID) with 2 mg (1 mg in 2 sites) or 0.2 mg (0.1 mg in 2 sites) of MERS-vaccine at the indicated timepoints. Naive control animals were not vaccinated. All animals were challenged with live MERS-CoV four weeks after their final immunization. Blood was collected at the indicated timepoints for immune analysis. FIG. 1B depicts representative results demonstrating vaccine induced antigen specific IFN-γ ELISPOT responses. PBMCs from each animal at each timepoint were stimulated with overlapping peptide pools covering the full-length MERS spike protein overnight and the number of cells secreting IFN-γ were counted. Group average spot forming units (SFU) per million cells are presented. Error bars are SEM. FIG. 1C depicts representative results demonstrating MERS-CoV DNA vaccine induced antigen specific IFN-γ ELISPOT responses. PBMCs from each animal at each timepoint were stimulated with recombinant full-length MERS Spike protein overnight and the number of cells secreting IFN-γ were counted. Individual values are shown by the symbols with the group average indicated by the bar. Error bars are SEM. Open triangles, squares, diamonds, and inverted triangles depict the total T cell responses for animals that were not selected for challenge. FIG. 1D depicts representative results demonstrating vaccine induced MERS spike-specific endpoint binding titers. Sera from each animal at each timepoint was evaluated for its ability to bind to full-length MERS Spike, S1, S2, and RBD protein. Endpoint titers for individual animals are shown with the geometric mean and 95% confidence interval indicated by the bars. Open triangles, squares, diamonds, and inverted triangles depict the total T cell responses for animals that were not selected for challenge. FIG. 1E depicts representative results demonstrating vaccine-induced neutralizing antibody titers in animals selected for challenge (n=4/vaccinated groups, n=6/naive). Sera was evaluated for its ability to neutralize live MERS-CoV. Reciprocal neutralizing antibody (nAb) titers are shown with the box indicating 25^{th} percentile, median, and 75^{th} percentile and whiskers showing the min and max values.

FIG. 2, comprising FIG. 2A through FIG. 2E, depicts representative results demonstrating that post-challenge pathology was prevented by MERS-CoV DNA vaccine. FIG. 2A depicts representative clinical scores for each group post challenge. Animals were scored for visible signs of disease daily following challenge with increasing scores indicating more severe symptoms. FIG. 2B depicts representative viral loads in vaccinated vs naive animals. FIG. 2C depicts representative viral loads in various tissues for each group post challenge. The viral load at day 6 post challenge in respiratory tissues and lymph nodes was measured by RT-PCR. Individual animals are included in the box-and-whisker plots, with whiskers showing the minimum and maximum values. FIG. 2D depicts representative viral loads in various tissues for each group post challenge. The viral load at day 6 post challenge in respiratory tissues and lymph nodes was measured by RT-PCR. Individual animals are included in the box-and-whisker plots, with whiskers showing the minimum and maximum values. FIG. 2E depicts representative H&E stained and IHC stained lung tissue sections from animals in each vaccination group day 6 post challenge. Vaccinated animals demonstrated essentially normal lung parenchyma. The naive animal showed moderate interstitial pneumonia. Viral antigen was detected by IHC (pink stain) in 4/6 control animals, but none of the immunized animals. (*p<0.05, **p<0.01, ***p<0.001 compared to the naive control). Original magnification, ×40 (H&E, left); ×200 (H&E, right); ×400 (IHC).

FIG. 3, comprising FIG. 3A through FIG. 3C, depicts representative results demonstrating that serum cytokine changed post challenge. Individual values are shown by the symbols with the group average indicated by the bar with error bars showing SEM. (*p<0.05, **p<0.01, ***p<0.001 compared to the naive control). FIG. 3A depicts representative MCP-1 cytokine levels in serum post-challenge. FIG. 3B depicts representative IL-1ra cytokine levels in serum post-challenge. FIG. 3C depicts representative IL-15 cytokine levels in serum post-challenge.

FIG. 4 depicts a representative description of clinical signs of disease from all animals post challenge (Clinical signs of disease: Green=normal animals, yellow=moderate, red=severe).

FIG. 5 depicts representative results of total peptide stimulated antigen-specific IFN-γ ELISPOT responses as described in FIG. 1. The total peptide stimulated IFNγ ELISPOT response for each animal is shown by the symbols with the group average indicated by the bar. Error bars are SEM. Open symbols depict the responses for animals that were not selected for challenge.

FIG. 6, comprising FIG. 6A through FIG. 6B, depict representative p values for lung viral loads in vaccinated and control animals. FIG. 6A depicts representative p values for lung viral loads between all vaccinated and control animals (corrected for multiple comparisons, p<0.05 is considered significant. Blue=significant) (as depicted in FIG. 2B). Parametric t-test, adjusted for multiple comparisons using a Bonferroni correction, was used for statistical analysis. FIG. 6B depicts representative p values for lung viral loads between vaccine groups and control animals (corrected for multiple comparisons, p<0.05 is considered significant. Blue=significant) (as depicted in FIGS. 2C and 2D). Parametric t-test, adjusted for multiple comparisons using a Bonferroni correction, was used for statistical analysis.

### DETAILED DESCRIPTION OF THE INVENTION

The present invention is based, in part, on the unexpected result that intradermal administering of an immunogenic composition and/or vaccine against MERS-CoV induced faster seroconversion and higher binding antibody titers at lower dose. Thus, the present invention relates, in part, to an intradermal vaccine comprising a MERS-CoV antigen.

In one embodiment, the MERS-CoV antigen is a MERS-CoV consensus spike antigen. In one embodiment, the MERS-CoV consensus spike antigen is derived from the sequences of spike antigens from strains of MERS-CoV, and thus, the MERS-CoV consensus spike antigen is unique. In one embodiment, the MERS-CoV consensus spike antigen lacks a cytoplasmic domain. Accordingly, in various aspects of the present invention, the intradermal vaccine of the present invention is widely applicable to multiple strains of MERS-CoV because of the unique sequences of the MERS-CoV consensus spike antigen. These unique sequences allow the vaccine to be universally protective against multiple strains of MERS-CoV, including genetically diverse variants of MERS-CoV.

In one aspect of the present invention, the intradermal vaccine is used to protect against and treat any number of strains of MERS-CoV. In one aspect of the present invention, the intradermal vaccine elicits both humoral and cellular immune responses that target the MERS-CoV spike antigen. In one aspect of the present invention, the intradermal vaccine elicits neutralizing antibodies and immunoglobulin G (IgG) antibodies that are reactive with the MERS-CoV spike antigen. In another aspect of the present invention, the intradermal vaccine elicits CD8⁺ and CD4⁺ T cell responses that are reactive to the MERS-CoV spike antigen and produce interferon-gamma (IFN-γ), tumor necrosis factor alpha (TNF-α), and interleukin-2 (IL-2).

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The singular forms “a,” “and” and “the” include plural references unless the context clearly dictates otherwise.

The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The present disclosure also contemplates other embodiments “comprising,” “consisting of” and “consisting essentially of,” the embodiments or elements presented herein, whether explicitly set forth or not.

“Adjuvant” as used herein means any molecule added to the vaccine described herein to enhance the immunogenicity of the antigen.

“Antibody” as used herein means an antibody of classes IgG, IgM, IgA, IgD or IgE, or fragments, fragments or derivatives thereof, including Fab, F(ab′)₂, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below.

“Fragment” or “immunogenic fragment” with respect to polypeptide sequences means a polypeptide capable of eliciting an immune response in a mammal that cross reacts with a full length wild type strain MERS-CoV antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of a consensus protein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a MERS-CoV protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Subject” as used herein can mean a mammal that wants to or is in need of being immunized with the herein described vaccine. The mammal can be a human, chimpanzee, dog, cat, horse, cow, mouse, or rat.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A “variant” may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Vaccine

The present invention provides immunogenic compositions for intradermal delivery, such as intradermal vaccines, comprising a Middle East Respiratory Syndrome coronavirus (MERS-CoV) antigen, a fragment thereof, a variant thereof, or a combination thereof. In one embodiment, the intradermal vaccine is used to protect against any number of strains of MERS-CoV, thereby treating, preventing, and/or protecting against MERS-CoV based pathologies. In one embodiment, the intradermal vaccine induces an immune response of a subject administered the vaccine intradermally, thereby protecting against and treating MERS-CoV infection.

In one embodiment, the vaccine is administered intradermally. In some embodiments, the vaccine is further administered via electroporation, or injection, or subcutaneously, or intramuscularly.

In one aspect, the intradermal vaccine is a DNA vaccine, a peptide vaccine, or a combination DNA and peptide vaccine.

In one embodiment, the DNA vaccine comprises a nucleic acid sequence encoding the MERS-CoV antigen. In some embodiments, the nucleic acid sequence is DNA, RNA, cDNA, a variant thereof, a fragment thereof, or a combination thereof. In some embodiments, the nucleic acid sequence further includes additional sequences that encode linker, leader, or tag sequences that are linked to the MERS-CoV antigen by a peptide bond.

In some embodiments, the peptide vaccine comprises a MERS-CoV antigenic peptide, a MERS-CoV antigenic protein, a variant thereof, a fragment thereof, or a combination thereof.

In some embodiments, the combination DNA and peptide vaccine comprises the above described nucleic acid sequence encoding the MERS-CoV antigen and the MERS-CoV antigenic peptide or protein, in which the MERS-CoV antigenic peptide or protein and the encoded MERS-CoV antigen have the same amino acid sequence.

In one aspect of the present invention, the vaccine induces a humoral immune response in the subject administered the vaccine intradermally. In one embodiment, the induced humoral immune response is specific for the MERS-CoV antigen. In one embodiment, the induced humoral immune response is reactive with the MERS-CoV antigen.

In some embodiments, the humoral immune response is induced in the subject administered the vaccine intradermally by about 1.5-fold to about 16-fold, about 2-fold to about 12-fold, or about 3-fold to about 10-fold. In some embodiments, the humoral immune response is induced in the subject administered the vaccine intradermally by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

In one embodiment, the humoral immune response induced by the vaccine comprises an increased level of neutralizing antibodies associated with the subject administered the vaccine intradermally as compared to a subject not administered the vaccine. In one embodiment, the humoral immune response induced by the vaccine comprises an increased level of neutralizing antibodies associated with the subject administered the vaccine intradermally as compared to a subject administered the vaccine intramuscularly. In one embodiment, the neutralizing antibodies are specific for the MERS-CoV antigen. In one embodiment, the neutralizing antibodies are reactive with the MERS-CoV antigen. In one embodiment, the neutralizing antibodies provide protection against and/or treatment of MERS-CoV infection and its associated pathologies in the subject administered the vaccine.

In one embodiment, the humoral immune response induced by the vaccine comprises an increased level of IgG antibodies associated with the subject administered the vaccine intradermally as compared to a subject not administered the vaccine. In one embodiment, the humoral immune response induced by the vaccine comprises an increased level of IgG antibodies associated with the subject administered the vaccine intradermally as compared to a subject administered the vaccine intramuscularly. In one embodiment, the IgG antibodies are specific for the MERS-CoV antigen. In one embodiment, the IgG antibodies are reactive with the MERS-CoV antigen.

In one embodiment, the humoral response is cross-reactive against two or more strains of the MERS-CoV. In some embodiments, the level of IgG antibody associated with the subject administered the vaccine intradermally is increased by about 1.5-fold to about 16-fold, about 2-fold to about 12-fold, or about 3-fold to about 10-fold as compared to the comparator (e.g., the subject not administered the vaccine or the subject administered the vaccine intramuscularly). In some embodiments, the level of IgG antibody associated with the subject administered the vaccine intradermally is increased by at least about 1.1-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the comparator (e.g., the subject not administered the vaccine or the subject administered the vaccine intramuscularly).

In one embodiment, the vaccine induces a cellular immune response in the subject administered the vaccine intradermally. In one embodiment, the induced cellular immune response is specific for the MERS-CoV antigen. In one embodiment, the induced cellular immune response is reactive to the MERS-CoV antigen. In one embodiment, the cellular response is cross-reactive against two or more strains of the MERS-CoV.

In one embodiment, the induced cellular immune response comprises eliciting a CD8⁺ T cell response. In one embodiment, the elicited CD8⁺ T cell response is reactive with the MERS-CoV antigen. In one embodiment, the elicited CD8⁺ T cell response is polyfunctional. In some embodiments, the induced cellular immune response comprises eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

In one embodiment, the induced cellular immune response comprises an increased CD8⁺ T cell response associated with the subject administered the vaccine intradermally as compared to the subject not administered the vaccine. In one embodiment, the induced cellular immune response comprises an increased CD8⁺ T cell response associated with the subject administered the vaccine intradermally as compared to the subject administered the vaccine intramuscularly.

In some embodiments, the CD8⁺ T cell response associated with the subject administered the vaccine intradermally is increased by about 2-fold to about 30-fold, about 3-fold to about 25-fold, or about 4-fold to about 20-fold as compared to the comparator (e.g., the subject not administered the vaccine or the subject administered the vaccine intramuscularly). In some embodiments, the CD8⁺ T cell response associated with the subject administered the vaccine intradermally is increased by at least about 1.1-fold, at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the comparator (e.g., the subject not administered the vaccine or the subject administered the vaccine intramuscularly).

In one embodiment, the induced cellular immune response comprises an increased frequency of CD3⁺CD8⁺ T cells that produce IFN-γ. In some embodiments, the frequency of CD3⁺CD8⁺IFN-γ⁺ T cells associated with the subject administered the vaccine intradermally is increased by at least about 1.1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the comparator (e.g., the subject not administered the vaccine or the subject administered the vaccine intramuscularly).

In one embodiment, the induced cellular immune response comprises an increased frequency of CD3⁺CD8⁺ T cells that produce TNF-α. In some embodiments, the frequency of CD3⁺CD8⁺TNF-α⁺ T cells associated with the subject administered the vaccine intradermally is increased by at least about 1.1-fold, 2-fold, 3-fold, 4-fold, 5-fold, 6-fold, 7-fold, 8-fold, 9-fold, 10-fold, 11-fold, 12-fold, 13-fold, or 14-fold as compared to the comparator (e.g., the subject not administered the vaccine or the subject administered the vaccine intramuscularly).

In one embodiment, the induced cellular immune response comprises an increased frequency of CD3⁺CD8⁺ T cells that produce IL-2. In some embodiments, the frequency of CD3⁺CD8⁺IL-2⁺ T cells associated with the subject administered the vaccine intradermally is increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the comparator (e.g., the subject not administered the vaccine or the subject administered the vaccine intramuscularly).

In one embodiment, the induced cellular immune response comprises an increased frequency of CD3⁺CD8⁺ T cells that produce both IFN-γ and TNF-α. In some embodiments, the frequency of CD3⁺CD8⁺IFN-γ⁺TNF-α⁺ T cells associated with the subject administered the vaccine intradermally is increased by at least about 1.1-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the comparator (e.g., the subject not administered the vaccine or the subject administered the vaccine intramuscularly).

In one embodiment, the cellular immune response induced by the intradermal vaccine comprises eliciting a CD4⁺ T cell response. In one embodiment, the elicited CD4⁺ T cell response is reactive with the MERS-CoV antigen. In one embodiment, the elicited CD4⁺ T cell response is polyfunctional. In one embodiment, the induced cellular immune response comprises eliciting a CD4⁺ T cell response, in which the CD4⁺ T cells produce IFN-γ, TNF-α, IL-2, or a combination of IFN-γ and TNF-α.

In one embodiment, the induced cellular immune response comprises an increased frequency of CD3⁺CD4⁺ T cells that produce IFN-γ. In some embodiments, the frequency of CD3⁺CD4⁺IFN-γ⁺ T cells associated with the subject administered the vaccine intradermally is increased by at least about 1.1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the comparator (e.g., the subject not administered the vaccine or the subject administered the vaccine intramuscularly).

In one embodiment, the induced cellular immune response comprises an increased frequency of CD3⁺CD4⁺ T cells that produce TNF-α. In some embodiments, the frequency of CD3⁺CD4⁺TNF-α⁺ T cells associated with the subject administered the vaccine intradermally is increased by at least about 1.1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the comparator (e.g., the subject not administered the vaccine or the subject administered the vaccine intramuscularly).

In one embodiment, the induced cellular immune response comprises an increased frequency of CD3⁺CD4⁺ T cells that produce IL-2. In some embodiments, the frequency of CD3⁺CD4⁺IL-2⁺ T cells associated with the subject administered the vaccine intradermally is increased by at least about 1.1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the comparator (e.g., the subject not administered the vaccine or the subject administered the vaccine intramuscularly).

In one embodiment, the induced cellular immune response comprises an increased frequency of CD3⁺CD4⁺ T cells that produce both IFN-γ and TNF-α. In some embodiments, the frequency of CD3⁺CD4⁺IFN-γ⁺TNF-α⁺ associated with the subject administered the vaccine intradermally is increased by at least about 1.1-fold, 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the comparator (e.g., the subject not administered the vaccine or the subject administered the vaccine intramuscularly).

In various aspects of the present invention, the vaccine comprises features required of effective vaccines, such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

a. Middle East Respiratory Syndrome Coronavirus (MERS-CoV) Antigen

As described above, in various embodiments, the vaccine of the present invention comprises a MERS-CoV antigen, a fragment thereof, a variant thereof, or a combination thereof. Coronaviruses, including MERS-CoV, are encapsulated by a membrane and have a type 1 membrane glycoprotein known as spike (S) protein, which forms protruding spikes on the surface of the coronavirus. The spike protein facilitates binding of the coronavirus to proteins located on the surface of a cell, for example, the metalloprotease amino peptidase N, and mediates cell-viral membrane fusion. In particular, the spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion. Thus, in various aspects of the present invention, the MERS-CoV antigen comprise a MERS-CoV spike protein, a S1 subunit of a MERS-CoV spike protein, or a S2 subunit of a MERS-CoV spike protein.

Upon binding cell surface proteins and membrane fusion, the coronavirus enters the cell and its singled-stranded RNA genome is released into the cytoplasm of the infected cell. The singled-stranded RNA genome is a positive strand and thus, can be translated into a RNA polymerase, which produces additional viral RNAs that are minus strands. Thus, in one embodiment, the MERS-CoV antigen is a MERS-CoV RNA polymerase.

The viral minus RNA strands are transcribed into smaller, subgenomic positive RNA strands, which are used to translate other viral proteins, for example, nucleocapsid (N) protein, envelope (E) protein, and matrix (M) protein. Thus, in various embodiments, the MERS-CoV antigen comprises a MERS-CoV nucleocapsid protein, a MERS-CoV envelope protein, or a MERS-CoV matrix protein.

In various embodiment, the viral minus RNA strands can also be used to replicate the viral genome, which is bound by nucleocapsid protein. Matrix protein, along with spike protein, is integrated into the endoplasmic reticulum of the infected cell. Together, the nucleocapsid protein bound to the viral genome and the membrane-embedded matrix and spike proteins are budded into the lumen of the endoplasmic reticulum, thereby encasing the viral genome in a membrane. The viral progeny are then transported by golgi vesicles to the cell membrane of the infected cell and released into the extracellular space by endocytosis.

In some embodiments, the MERS-CoV antigen is a MERS-CoV spike protein, a MERS-CoV RNA polymerase, a MERS-CoV nucleocapsid protein, a MERS-CoV envelope protein, a MERS-CoV matrix protein, a fragment thereof, a variant thereof, or a combination thereof. The MERS-CoV antigen can be a consensus antigen derived from two or more MERS-CoV spike antigens, two or more MERS-CoV RNA polymerases, two or more MERS-CoV nucleocapsid proteins, two or more envelope proteins, two or more matrix proteins, or a combination thereof.

In some embodiments, the MERS-CoV consensus antigen is modified for improved expression. In some embodiments, the modification includes codon optimization, RNA optimization, addition of a kozak sequence for increased translation initiation, and/or the addition of an immunoglobulin leader sequence to increase the immunogenicity of the MERS-CoV antigen. In some embodiments the MERS-CoV antigen includes an IgE leader, which can be the amino acid sequence set forth in SEQ ID NO:6 and encoded by the nucleotide sequence set forth in SEQ ID NO:5.

(1) MERS-CoV Spike Antigen

In various aspects of the present invention, the MERS-CoV antigen is a MERS-CoV spike antigen, a fragment thereof, a variant thereof, or a combination thereof. In one aspect, the MERS-CoV spike antigen is capable of eliciting an immune response in a mammal against one or more MERS-CoV strains. In one aspect, the MERS-CoV spike antigen comprises an epitope(s) that makes it particularly effective as an immunogen against which an anti-MERS-CoV immune response can be induced.

In one embodiment, the MERS-CoV spike antigen is a consensus sequence derived from two or more strains of MERS-CoV. In some embodiments, the MERS-CoV spike antigen comprises a consensus sequence and/or modification(s) for improved expression. Examples of such modifications include, but are not limited to codon optimization, RNA optimization, addition of a kozak sequence for increased translation initiation, and/or the addition of an immunoglobulin leader sequence to increase the immunogenicity of the MERS-CoV spike antigen.

In some embodiments, the MERS-CoV consensus spike antigen comprises a signal peptide, such as an immunoglobulin signal peptide, for example, but not limited to, an immunoglobulin E (IgE) or immunoglobulin (IgG) signal peptide. In one embodiment, the MERS-CoV consensus spike antigen comprises a hemagglutinin (HA) tag. In some embodiments, the MERS-CoV consensus spike antigen is designed to elicit stronger and broader cellular and/or humoral immune responses than a corresponding codon optimized spike antigen.

In one embodiment, the MERS-CoV consensus spike antigen is the nucleic acid sequence SEQ ID NO:1, which encodes SEQ ID NO:2. In some embodiments, the MERS-CoV consensus spike antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity over an entire length of the nucleic acid sequence set forth in SEQ ID NO:1. In other embodiments, the MERS-CoV consensus spike antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

In one embodiment, the MERS-CoV consensus spike antigen is the amino acid sequence SEQ ID NO:2. In some embodiments, the MERS-CoV consensus spike antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

In one embodiment, immunogenic fragments of SEQ ID NO:2 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:2. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:2 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:2. Some embodiments relate to immunogenic fragments that have 96% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:1. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:1. Immunogenic fragments can be at least 95%, at least 96%, at least 97% at least 98% or at least 99% homologous to fragments of SEQ ID NO:1. In some embodiments, immunogenic fragments include sequences that encode a leader sequence, such as for example an immunoglobulin leader, such as the IgE leader. In some embodiments, fragments are free of coding sequences that encode a leader sequence.

(a) MERS-CoV Spike Antigen lacking a Cytoplasmic Domain

In various aspects of the present invention, the MERS-CoV antigen is a MERS-CoV spike antigen lacking a cytoplasmic domain (i.e., also referred to herein as “MERS-CoV spike antigen ΔCD”), a fragment thereof, a variant thereof, or a combination thereof. In one embodiment, the MERS-CoV spike antigen ΔCD is capable of eliciting an immune response in a mammal against one or more MERS-CoV strains. In one embodiment, the MERS-CoV spike antigen ΔCD comprises an epitope(s) that makes it particularly effective as an immunogen against which an anti-MERS-CoV immune response can be induced.

In one embodiment, the MERS-CoV spike antigen ΔCD is a consensus sequence derived from two or more strains of MERS-CoV. In some embodiments, the MERS-CoV spike antigen ΔCD comprises a consensus sequence and/or modification(s) for improved expression. Examples of such modification include, but are not limited to codon optimization, RNA optimization, addition of a kozak sequence for increased translation initiation, and/or the addition of an immunoglobulin leader sequence to increase the immunogenicity of the MERS-CoV spike antigen ΔCD.

In some embodiments, the MERS-CoV consensus spike antigen ΔCD comprises a signal peptide, such as an immunoglobulin signal peptide, for example, but not limited to, an immunoglobulin E (IgE) or immunoglobulin (IgG) signal peptide. In some embodiments, the consensus spike antigen ΔCD comprises a hemagglutinin (HA) tag. In some embodiments, the MERS-CoV consensus spike antigen ΔCD is designed to elicit stronger and broader cellular and/or humoral immune responses than a corresponding codon optimized spike antigen ΔCD.

In one embodiment, the MERS-CoV consensus spike antigen ΔCD is the nucleic acid sequence SEQ ID NO:3, which encodes SEQ ID NO:4. In some embodiments, the MERS-CoV consensus spike antigen ΔCD can be the nucleic acid sequence having at least about 90%, 91%, 92%, 93%, 94%, 95%, 96%, 97%, 98%, 99% or 100% identity over an entire length of the nucleic acid sequence set forth in SEQ ID NO:3. In other embodiments, the MERS-CoV consensus spike antigen ΔCD can be the nucleic acid sequence that encodes the amino acid sequence having at least about 90%, 91%, 92%, 93%, 94%, 95%, 96%, 97%, 98%, 99%, or 100% identity over an entire length of the amino acid sequence set forth in SEQ ID NO:4.

In one embodiment, the MERS-CoV consensus spike antigen ΔCD is the amino acid sequence SEQ ID NO:4. In some embodiments, the MERS-CoV consensus spike antigen ΔCD can be the amino acid sequence having at least about 90%, 91%, 92%, 93%, 94%, 95%, 96%, 97%, 98%, 99%, or 100% identity over an entire length of the amino acid sequence set forth in SEQ ID NO:4.

Immunogenic fragments of SEQ ID NO:4 can be provided. Immunogenic fragments can comprise at least 60%, at least 65%, at least 70%, at least 75%, at least 80%, at least 85%, at least 90%, at least 95%, at least 96%, at least 97%, at least 98% or at least 99% of SEQ ID NO:4. In some embodiments, immunogenic fragments include a leader sequence, such as for example an immunoglobulin leader, such as the IgE leader. In some embodiments, immunogenic fragments are free of a leader sequence.

Immunogenic fragments of proteins with amino acid sequences homologous to immunogenic fragments of SEQ ID NO:4 can be provided. Such immunogenic fragments can comprise at least 60%, at least 65%, at least 70%, at least 75%, at least 80%, at least 85%, at least 90%, at least 95%, at least 96%, at least 97%, at least 98% or at least 99% of proteins that are 95% homologous to SEQ ID NO:4. Some embodiments relate to immunogenic fragments that have 96% homology to the immunogenic fragments of consensus protein sequences herein. Some embodiments relate to immunogenic fragments that have 97% homology to the immunogenic fragments of consensus protein sequences herein. Some embodiments relate to immunogenic fragments that have 98% homology to the immunogenic fragments of consensus protein sequences herein. Some embodiments relate to immunogenic fragments that have 99% homology to the immunogenic fragments of consensus protein sequences herein. In some embodiments, immunogenic fragments include a leader sequence, such as for example an immunoglobulin leader, such as the IgE leader. In some embodiments, immunogenic fragments are free of a leader sequence.

Some embodiments relate to immunogenic fragments of SEQ ID NO:3. Immunogenic fragments can be at least 60%, at least 65%, at least 70%, at least 75%, at least 80%, at least 85%, at least 90%, at least 95%, at least 96%, at least 97%, at least 98% or at least 99% of SEQ ID NO:3. Immunogenic fragments can be at least 95%, at least 96%, at least 97% at least 98% or at least 99% homologous to fragments of SEQ ID NO:3. In some embodiments, immunogenic fragments include sequences that encode a leader sequence, such as for example an immunoglobulin leader, such as the IgE leader. In some embodiments, fragments are free of coding sequences that encode a leader sequence.

b. Vector

In various embodiments, the vaccine comprises one or more vectors that include a nucleic acid encoding the antigen. The one or more vectors can be capable of expressing the antigen. The vector can have a nucleic acid sequence containing an origin of replication. The vector can be a plasmid, bacteriophage, bacterial artificial chromosome or yeast artificial chromosome. The vector can be either a self-replicating extrachromosomal vector or a vector which integrates into a host genome.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

(1) Expression Vectors

The vector can be a circular plasmid or a linear nucleic acid. The circular plasmid and linear nucleic acid are capable of directing expression of a particular nucleotide sequence in an appropriate subject cell. The vector can have a promoter operably linked to the antigen-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or of an inducible promoter, which initiates transcription only when the host cell is exposed to some particular external stimulus. In the case of a multicellular organism, the promoter can also be specific to a particular tissue or organ or stage of development.

(2) Circular and Linear Vectors

The vector may be a circular plasmid, which may transform a target cell by integration into the cellular genome or exist extrachromosomally (e.g., autonomous replicating plasmid with an origin of replication).

The vector can be pVAX, pcDNA3.0, or provax, or any other expression vector capable of expressing DNA encoding the antigen and enabling a cell to translate the sequence to an antigen that is recognized by the immune system.

Also provided herein is a linear nucleic acid vaccine, or linear expression cassette (“LEC”), that is capable of being efficiently delivered to a subject via electroporation and expressing one or more desired antigens. The LEC may be any linear DNA devoid of any phosphate backbone. The DNA may encode one or more antigens. The LEC may contain a promoter, an intron, a stop codon, and/or a polyadenylation signal. The expression of the antigen may be controlled by the promoter. The LEC may not contain any antibiotic resistance genes and/or a phosphate backbone. The LEC may not contain other nucleic acid sequences unrelated to the desired antigen gene expression.

The LEC may be derived from any plasmid capable of being linearized. The plasmid may be capable of expressing the antigen. The plasmid can be pNP (Puerto Rico/34) or pM2 (New Caledonia/99). The plasmid may be WLV009, pVAX, pcDNA3.0, or provax, or any other expression vector capable of expressing DNA encoding the antigen and enabling a cell to translate the sequence to an antigen that is recognized by the immune system.

The LEC can be perM2. The LEC can be perNP. perNP and perMR can be derived from pNP (Puerto Rico/34) and pM2 (New Caledonia/99), respectively.

(3) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

c. Excipients and other Components of the Vaccine

The vaccine may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/mL. The transfection facilitating agent may also include surface active agents, such as immune-stimulating complexes (ISCOMS), Freunds incomplete adjuvant, LPS analog including monophosphoryl lipid A, muramyl peptides, quinone analogs and vesicles, such as squalene and squalene, and hyaluronic acid may also be used administered in conjunction with the genetic construct. The DNA plasmid vaccines may also include a transfection facilitating agent, such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/mL, less than 2 mg/mL, less than 1 mg/mL, less than 0.750 mg/mL, less than 0.500 mg/mL, less than 0.250 mg/mL, less than 0.100 mg/mL, less than 0.050 mg/mL, or less than 0.010 mg/mL.

The pharmaceutically acceptable excipient can be an adjuvant. The adjuvant can be other genes that are expressed in an alternative plasmid or are delivered as proteins in combination with the plasmid above in the vaccine. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Serial No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 3. Method of Vaccination

The present invention also provides a method of treating, protecting against, and/or preventing disease in a subject in need thereof by administering the vaccine intradermally to the subject. In various aspects of the present invention, intradermal administration of the vaccine to the subject induces or elicits an immune response in the subject. In some embodiments, the induced immune response is used to treat, prevent, and/or protect against disease, for example, pathologies relating to MERS-CoV infection. In one embodiment, the induced immune response provides the subject administered the vaccine resistance to one or more MERS-CoV strains.

In some embodiments, the induced immune response includes an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies that are reactive to the antigen. The induced cellular immune response can include a CD8⁺ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Administration

The vaccine can be formulated in accordance with standard techniques well known to those skilled in the pharmaceutical art. Such compositions can be administered intradermally in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered intradermally in an amount sufficient to induce an immune response. In therapeutic applications, the vaccines are administered intradermally to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine administered intradermally can be further administered in combination with methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. For example, in one embodiment, the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

In some embodiments, the vaccine is delivered intradermally and in a combination with other delivery routes. For example, in some embodiments, the vaccine is delivered intradermally and in combination with other parenteral administration, e.g., intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be further administered to muscle, or can be administered via subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can be a liquid preparation such as a suspension, syrup or elixir. The vaccine can also be a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be further administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

In various aspects of the invention, the vaccine is delivered intradermally via a minimally invasive electroporation device. The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the present invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### 4. Kit

The present invention also provides a kit, which can be used for treating a subject using the method of vaccination described above. In one embodiment, the kit comprises the intradermal vaccine of the present invention. In one embodiment, the kit comprises instructions for carrying out the vaccination method described above and/or how to use the kit. In one embodiment, the instructions included in the kit are affixed to packaging material or are included as a package insert. While instructions are typically written or printed materials, they are not limited to such. Any medium capable of storing instructions and communicating them to an end user is contemplated by this disclosure. Such media include, but are not limited to, electronic storage media (e.g., magnetic discs, tapes, cartridges), optical media (e.g., CD ROM), and the like. As used herein, the term “instructions” can include the address of an internet site which provides instructions.

The present invention has multiple aspects, illustrated by the following non-limiting examples.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present disclosure and practice the claimed methods. The following working examples therefore, specifically point out the preferred embodiments of the present disclosure, and are not to be construed as limiting in any way the remainder of the disclosure.

### Example 1

### Intradermal Delivery of a Synthetic Consensus DNA Vaccine Protects Rhesus Macaques from Middle East Respiratory Syndrome Coronavirus

DNA vaccines are a non-live, non-infectious platform that are re-administrable, easily scalable for manufacturing, have an established safety and tolerability profile and are heat-stable (Kutzler M A et al., 2008, Nat. Rev. Genet., 9:776-788; Lee L Y Y et al., 2018, Front. Immunol., 9:1568). The rapid development of an anti-MERS synthetic DNA vaccine encoding a full-length MERS-CoV Spike antigen, which induced robust humoral and cellular responses and protected rhesus macaques from MERS-CoV challenge, was previously described (Muthumani K et al., 2015, Sci. Transl. Med., 7:301ra132). This MERS DNA vaccine candidate (INO-4700/GLS-5300), delivered by intramuscular (IM) administration, was found to be safe and tolerable with a three-dose injection regimen in a recently completed human Phase I study (Modjarrad K et al., 2019, Lancet Infect. Dis., 19:1013-1022), and advanced to expanded studies of Phase I/IIa trial in South Korea (Saunders K O et al., 2015, J. Virol., 89:5895-5903).

Further study of low-dose delivery with shortened dosing regimens are important to rapidly induce protective immunity, particularly during an emerging outbreak as is an important consideration where herd immunity is limited (Bernasconi V et al., 2020, Bundesgesundheitsblatt Gesundheitsforschung Gesundheitsschutz, 63:65-73).

The present example describes delivery of the MERS DNA vaccine candidate INO-4700 employing an abbreviated 2-dose immunization regimen in rhesus macaques, immunogenicity and protective efficacy with a comparison of the DNA vaccine in an important protection model by intramuscular (IM) delivery and intradermal (ID) delivery. Induction of strong antibody titers against the full-length Spike protein as well as antigenic components of Spike protein, such as the receptor-binding domain (RBD), S1, and S2 regions, induction of neutralizing antibody responses, and cellular immune responses were observed. Finally, the animals were challenged and the impact of the vaccination on infection against vigorous MERS-CoV challenge in nonhuman primates (NHPs) was determined. Macaques receiving this 2-dose vaccine demonstrated lower viral loads with protection of the lung from inflammation, protection against elevated cytokine levels, and, most importantly, protection against clinical disease symptoms such as breathing difficulties. Even low-dose ID delivery afforded comparable efficacy to higher dose ID and IM regimens, and both ID immunizations exhibited improved disease control compared with IM vaccination. The data support further evaluation of simple dose-sparing ID-delivered DNA vaccination regimens against MERS-CoV. These advances have important applicability for this DNA vaccine delivery against other emerging betacoronaviruses, such as SARS-CoV-2, as well as for future emerging infectious diseases.

### Immunogenicity of Intradermal-Delivered MERS DNA Vaccine

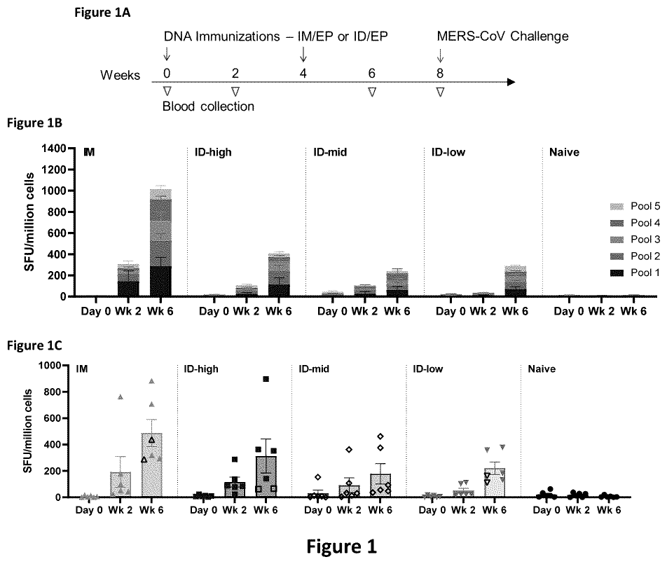

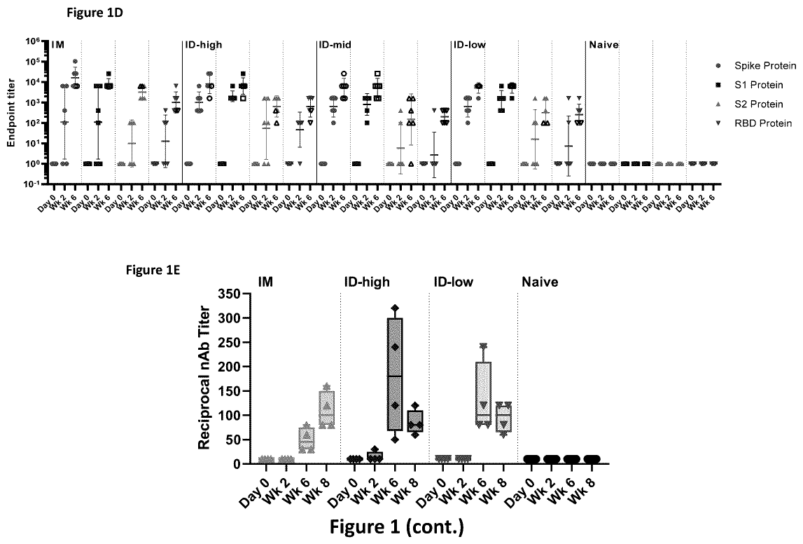

Very recent advances in formulations for ID delivery of synthetic DNA vaccines with adaptive electroporation (EP) have significantly improved the generation of antigen-specific immune responses, including long-term antibody and T cell responses induced in human trials, with responses persisting at least 1 year post vaccination (Tebas P et al., 2017, N. Engl. J. Med.; Tebas P et al., 2019, J. Infect. Dis., 220:400-410; DeRosa S et al., 2020, JCI Insight). ID delivery of DNA vaccines is tolerable, simple to administer, and is potentially more immunogenic than IM delivery when given at the same dose in recent clinical studies (Modjarrad K et al., 2019, Lancet Infect. Dis., 19:1013-1022; Tebas P et al., 2017, N. Engl. J. Med.; Tebas P et al., 2019, J. Infect. Dis., 220:400-410; Gardner M R et al., 2015, Nature, 519:87-91). Therefore, the efficacy of the previously described synthetic MERS DNA vaccine (Muthumani K et al., 2015, Sci. Transl. Med., 7:301ra132), which had been studied in NHP using an IM 3 dose immunization regime, was evaluated. The present efforts studied an abbreviated 2-dose ID immunization regimen and compared this approach with IM delivery. Rhesus macaques (n=6/group) were first administered either a 0.2 mg low dose (ID-low), a 1 mg dose (ID-mid), or a 2 mg dose (ID-high) of the MERS DNA vaccine by ID injection followed by adaptive EP. The IM group (n=6) received a 1.0 mg dose. All vaccinated groups received a 2-dose regimen, spaced at a 4-week interval (FIG. 1A). The control group (n=6) was not vaccinated.

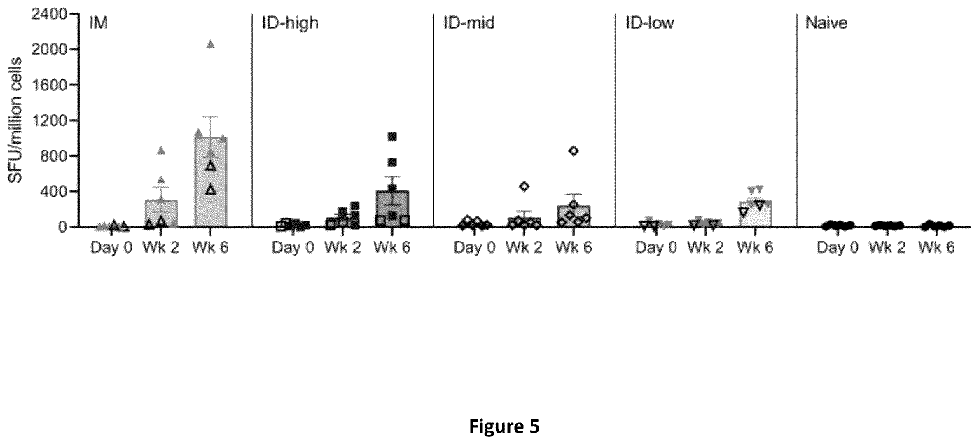

Cellular and humoral immune responses were assayed following each immunization. Following the immunization studies, 3 of the groups and 4 of the animals were selected from each of the selected groups for MERS viral challenge, based on space limitations. Both humoral and cellular responses were analyzed as the role of both adaptive immune compartments, which is likely to be important for virus clearance and recovery from infection as has been described for both SARS-CoV and MERS-CoV (Oh H L J et al., 2012, Emerg. Microbes Infect., 1:e23; Zhao J et al., 2017, Sci. Immunol., 2), and suggested by recent studies of human immune responses in convalescent SARS-CoV-2 patients (Grifoni A et al., 2020, Cell, 181:1489-1501; Robbiani D F et al., 2020, bioRxiv, 2020.2005.2013.092619). The induction of T cell responses by IFNγ ELISpot was analyzed two weeks after each immunization. T cell responses against peptide pools spanning the full-length S protein were readily detected in 6/6 NHPs in the IM group (432-2067 spot-forming units [SFU]/million PBMCs), 6/6 NHPs in the ID-high dose group (73-1018 SFU/million PBMCs), 6/6 NHPs in the ID-mid dose group (52-857 SFU/million PBMCs), 6/6 NHPs in the ID-low dose group (160-422 SFU/million PBMCs), and 0/6 NHPs in the naive control group (2-33 SFU/million PMBCs) after two DNA immunizations (FIG. 1B and FIG. 5). Additionally, IFNγ ELISpot T cell assays were performed using full-length recombinant Spike protein for stimulation as a tool to address rapid vaccine evaluation during an outbreak in the absence of synthetic peptide pools. Although fewer total spots were observed, on average, strong T cell responses induced in all groups following a similar trend to those observed with peptide pools (FIG. 1C), supporting the full-length antigen study as an additional assay tool in evaluation of vaccine immunogenicity.

To address the question of antibody responses following in vivo processing of a full-length Spike protein antigen, antibody endpoint titers against the full-length S as well as S1, S2, and receptor binding domains (RBD) by total IgG binding ELISA were assayed. After 1 immunization 67% (4/6) of IM animals, 100% (6/6) of ID-high animals, 100% (6/6) of ID-mid animals, and 100% (6/6) of ID-low animals seroconverted to full length S and S1 proteins. After 1 immunization 50% (3/6) of IM animals, 67% (4/6) of ID-high animals, 33% (2/6) of ID-mid animals, and 50% (3/6) of ID-low animals seroconverted to S2 protein. After 1 immunization 50% (3/6) of IM animals, 83% (5/6) of ID-high animals, 17% (1/6) of ID-mid animals, and 33% (2/6) of ID-low animals seroconverted to RBD protein. After 2 immunizations, all vaccinated animals seroconverted to full length S, S1, S2, and RBD proteins, except for 1 animal in the ID-mid group that did not seroconvert to S2 protein (FIG. 1D). Two weeks after the second immunization, geometric mean endpoint titers in all groups were approximately 10⁴ for both full length S and S1 proteins. Geometric mean endpoint titers in all groups were approximately 10²-10³ for S2 and RBD, with a trend for slightly higher titers in the IM group, though there were no significant differences in endpoint titer values between vaccine groups. Overall, the antibody responses induced in this study demonstrate the consistency of synthetic DNA vaccination and robust induction of antibody responses by the simple ID delivery and notably in the low dose (0.2 mg) ID-delivered MERS DNA vaccine (FIG. 1D).

Eighteen animals were selected for challenge with MERS-CoV of the 30 total animals, due to funding and space limitations. Based on the ELISPOT and endpoint binding antibody titer data available at the time, 12 total vaccinated animals and the 6 naive control animals that trended higher in each group were chosen to move into the challenge portion of the study (animals that were not selected for challenge are indicated by open shapes in FIGS. 1C and 1D). There is no statistical difference regarding immune responses between the animals in each group that were challenged compared with those that were not challenged. Because the ID-low group exhibited robust immunogenicity, its challenge outcome was compared to the ID-high group, so 4 animals each from the ID-high and ID-low groups were chosen for challenge. 4 animals from the IM group served as a comparison with previous studies, which were 3-dose immunization studies (Muthumani K et al., 2015, Sci. Transl. Med., 7:301ra132) (identified in FIG. 1C).

Neutralizing antibody titers for the challenged animals were assayed using MERS-CoV EMC/2012 (FIG. 1E). Neutralizing activity was detected in the sera after the boost, peaking at week 6, with average titers of 50, 170, and 130 for IM, ID-high, and ID-low groups, respectively. By week 8, all vaccinated groups had comparable neutralizing antibody titers, demonstrating that similar binding and neutralizing antibody titers could be induced by low dose (0.2 mg) ID vaccination as compared to higher doses (1.0 mg IM vaccination and 2.0 mg ID vaccination). Delivery i.d. appears dose sparing based on this comparison, and a similar observation has recently been reported for an HIV DNA vaccine studied in the clinic, which was delivered by the Cellectra i.d. EP approach (DeRosa S, et al., JCI Insight, 2020, 5(13): e137079).

### Challenge Outcome of ID vs IM MERS DNA Vaccine Regimens

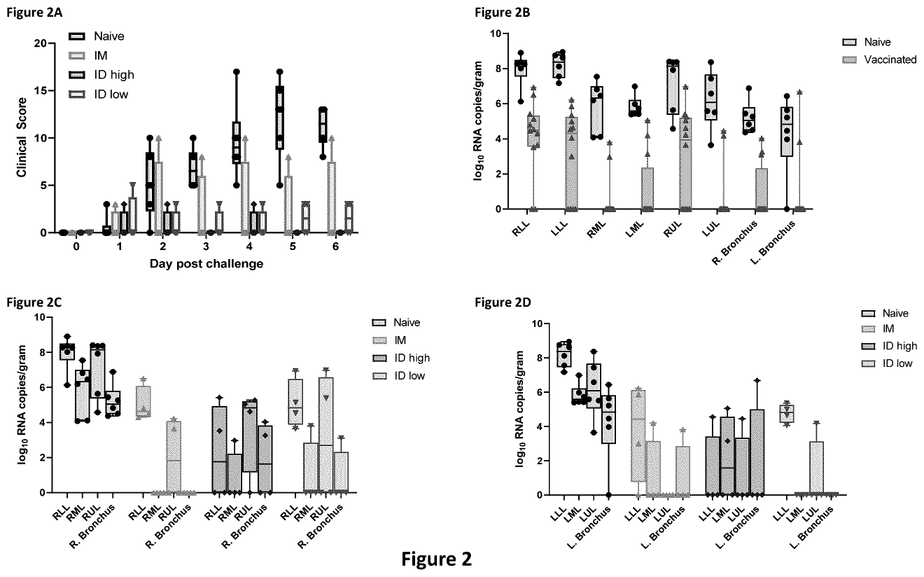

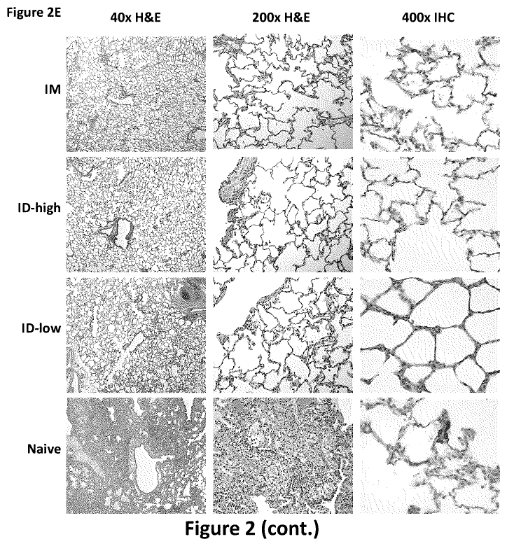

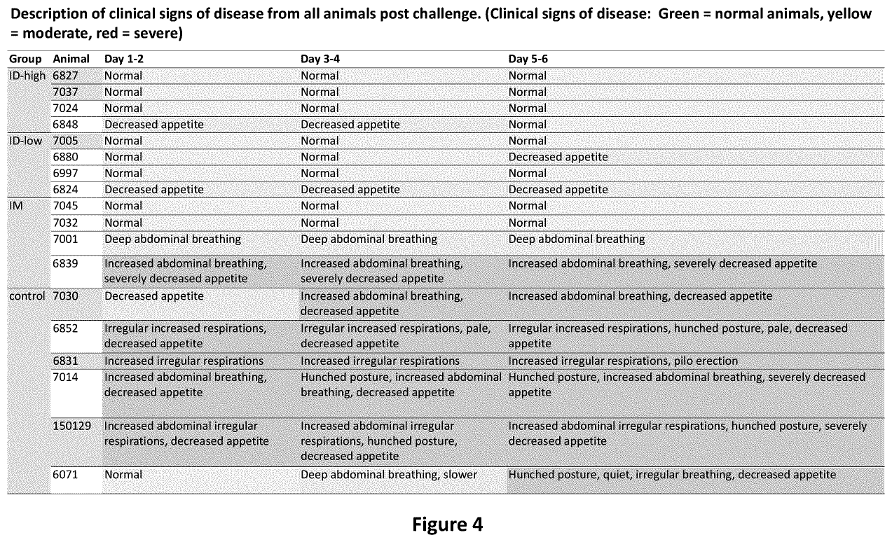

Macaques were challenged by inoculation with 7×10⁶ median tissue culture infectious dose (TCID₅₀) of MERS-CoV EMC/2012 strain through rigorous installation via a combination of intratracheal, intranasal, oral, and ocular administrations, as previously established (van Doremalen N et al., 2015, Antiviral Res., 122:28-38; de Wit E et al., 2013, Proc. Natl. Acad. Sci. USA, 110:16598-16603). NHPs were monitored for clinical signs of disease and also received chest X-rays on days 0, 1, 3, 5, and 6 post-challenge, before they were euthanized and necropsied on day 6 for lung pathology and viral load determination. All immunized animals, except one IM animal (11/12), had major reduction in clinical signs of disease as compared to the control group (FIG. 2A and FIG. 4), showing significant disease protection.

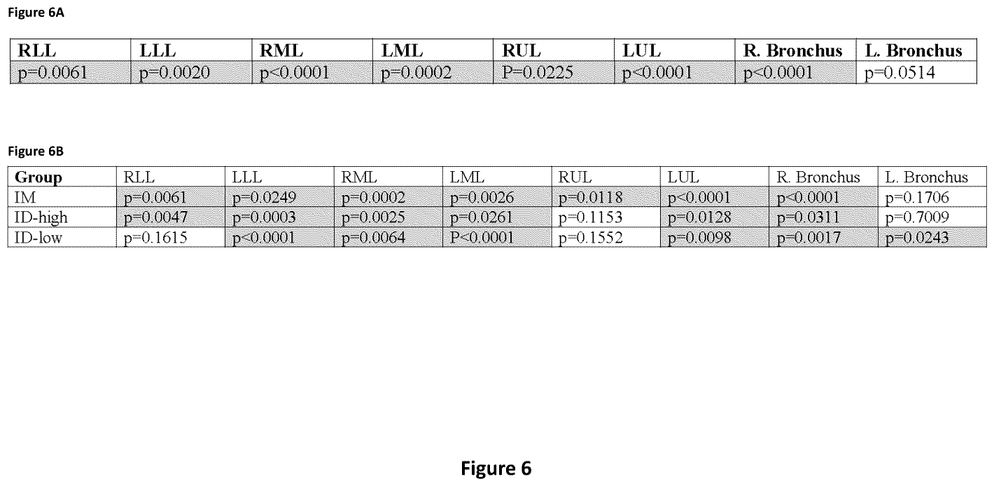

A upE qRT-PCR assay was performed to detect viral loads present in the collected lung tissue. Overall, compared to the unvaccinated animals, all MERS DNA vaccine groups exhibited log reductions in viral loads across all regions of the lower airways (FIG. 2B). Significantly decreased viral loads were observed in all vaccinated groups in the right bronchus, right middle lung, right lower lung, left upper lung, left middle lung, and left lower lung lobes (P values are listed in FIG. 6A). Four of four ID-low and three of four IM and ID-high animals had no viral loads in the left bronchus and animals trended towards decreased loads in the right upper lung (FIG. 2C and FIG. 2D; FIG. 6B).

Minimal virus was detected in the routes of installation challenge. It is likely that residual virus from the installation was being detected in these tissues as two animals were still positive in the conjunctiva (ocular administration route), a non-respiratory tissue, at day 6. In both the vaccinated and control groups, radiographic signs of disease were minimal. Lung tissues from all challenged animals were examined with H&E staining and immunohistochemistry (IHC) against MERS-CoV antigen to evaluate virus induced pathology (FIG. 2E). Histological evidence of mild focal interstitial pneumonia was observed in 5 out of 12 animals in the vaccinated group with multifocal moderate interstitial pneumonia in all 6 naive macaques. All 6 animals in the control group eventually developed multiple symptoms of disease, including difficulty breathing, as did 1 animal in the IM vaccinated group. No animals in the ID groups exhibited any symptoms associated with lung impact in the challenge course of the study. All of the control animals showed lung disease symptoms during the challenge course as well as other symptoms.

Finally, MERS-CoV antigen was detected through immunohistochemistry in 4/6 lung specimens from unvaccinated macaques but was not observed in any vaccinated macaques (FIG. 2F).

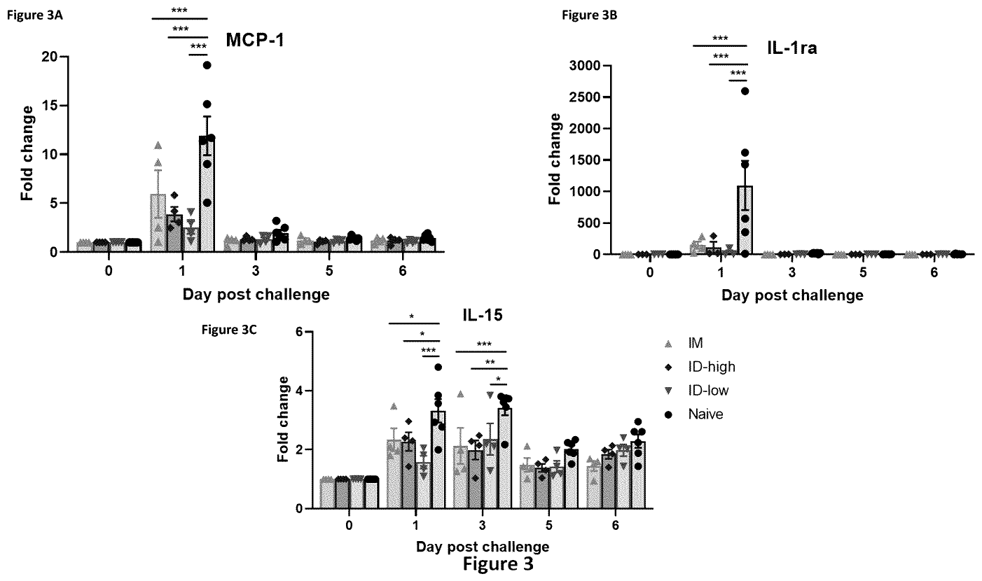

After challenge, sera was screened against a Luminex 23-cytokine panel (G-CSF, GM-CSF, IFNγ, IL1-β, IL-1ra, IL-2, IL-4, IL-5, IL-6, IL-8, IL-10, IL-12/23p40, IL-13, IL-15, IL-17A, IL-18, MCP-1, MIP-1α, sCD40L, TGFα, TNFα, VEGF) to assess potential inflammation impact. In control animals, significant increase of early innate cytokines MCP-1, IL-1ra, and IL-15 was observed. By comparison, this increase was abrogated in all vaccinated animals compared to unvaccinated controls (FIG. 3). No significant changes in other cytokines were observed or were below the limit of detection of the assay, supporting a lack of inflammation enhancement by this panel of immune markers.

In the last twenty years, three new CoV have emerged from zoonotic reservoirs (MERS, SARS, SARS-CoV-2); there are no licensed vaccines to prevent coronavirus infections in people. Vaccine candidates that are simple to deliver, well-tolerated, do not induce anti-vector immunity, and that can be readily administered in resource limited settings are advantageous. There have been a few other vaccine candidates studied in NHP challenge for MERS. These include an rRBD-plus-adjuvant-vaccine approach using 3 immunizations, which induced partial protection in a short term, 3 day challenge NHP model (Lan J et al., 2015, EBioMedicine, 2:1438-1446). A study using combinations of DNA vaccines and protein boosts showed limited vaccine impact on infection by CT scan read out (Wang L et al., 2015, Nat. Commun., 6:7712), and a different study tested a MERS-CoV spike recombinant Chimpanzee Ad vaccine (v. Doremalen N et al., 2020, Science Advances, 6:eaba8399) in a similar challenge model to the one presented, and similar to the earlier IM DNA immunization challenge (Muthumani K et al., 2015, Sci. Transl. Med., 7:301ra132). The vaccine was tested in one or two dose regimens. Both doses could impact viral disease and viral load, particularly in the lower respiratory tract, with the single dose regime exhibiting a smaller protective effect with limited impact on pathogenesis than the 2-dose regimen. Data from these reports and this present study are illustrative of the utility of this particular multiple route challenge NHP model developed at Rocky Mountain Labs (RML) for vaccine testing. It is reproducible and provides broad tissue sampling as well as disease read outs (Muthumani K et al., 2015, Sci. Transl. Med., 7:301ra132; van Doremalen N et al., 2015, Antiviral Res., 122:28-38; de Wit E et al., 2013, Proc. Natl. Acad. Sci. USA, 110:16598-16603; v. Doremalen N et al., 2020, Science Advances, 6:eaba8399).

The present study investigated the immunogenicity and protective efficacy of an ID-delivered synthetic MERS DNA vaccine using a shortened 2 dose immunization schedule and compared this to an IM delivered 2 dose DNA vaccine formulation. Immune analysis compared three different vaccine doses for their immune potency by ID delivery in parallel with IM delivery. The MERS DNA vaccines induced antibody responses against all regions of the Spike protein and robust neutralizing antibodies. Cellular immune responses were induced in all animals, which are likely to be important for clearance of virally infected cells, limiting pathogenesis, and reducing viral loads.

Vaccines that drive both antibody and T cell immunity could be important for preventing asymptomatic spread and protecting the lower airway, thus mitigating disease. For challenge, animals were downselected from three vaccine groups: the ID-low, ID-high, and IM groups. Challenge outcome showed that all three vaccination groups protected rhesus macaques against MERS-CoV EMC/2012 challenge compared to unvaccinated control animals; however, the i.d. groups, including the low-dose group, appeared to have the most robust effect on disease and symptomology. The present data extended the earlier work with IM delivered vaccines, including DNA vaccine to support investigation of ID-delivery of this MERS DNA vaccine candidate as well as abbreviated immunization regimens to rapidly induce protective immune responses, especially for emerging viral pathogens including novel Coronaviruses.

Moreover, the present example showed the first demonstration of protection with an ID-delivered MERS, or other coronavirus, vaccine candidate. Other MERS vaccine candidates have focused primarily on IM delivery (Lan j et al., 2015, EBioMedicine, 2:1438-1446; Wang L et al., 2015, Nat. Commun., 6:7712; Wang C et al., 2017, Oncotarget., 8:12686-12694). Furthermore, minimal signs of disease and pathology using a prime-boost ID-delivery regimen were observed. Using the same sensitive RT-PCR assay, significant decreases in viral loads in vaccinated animals in the lower lung regions were shown and significant reduction in early inflammatory cytokines in response to viral infection were achieved by a simple 2 dose regime. Additionally, this study demonstrated that a 2-dose regimen and the low-dose ID delivery was more impactful on disease than a higher dose IM delivery.

In this study, it was observed that IM delivery of synthetic DNA vaccines drives higher cellular immune responses than ID delivery at the same dose, though ID delivery is still able to induce consistent IFN-γ enzyme-linked immunospot (ELISPOT) responses. In contrast, ID delivery appeared to induce faster seroconversion, and in many cases, higher binding antibody titers and neutralizing antibody titers than IM delivery. This trend can be seen in this study with a MERS-CoV Spike DNA vaccine (FIG. 1), as well as in recent clinical studies of DNA vaccines targeting HIV (DeRosa S et al., 2020, JCI Insight) and Ebola (Tebas P et al., 2019, J. Infect. Dis., 220:400-410). In addition, it is likely that there is a different induction of T cell trafficking induced by ID vs IM immunization such as has recently been reported in a leishmania model system (Louis L et al., 2019, Infect. Immure., 87, 1-14). One hypothesis is that different cell populations are transfected between the muscle (myocytes) and skin (keratinocytes, fibroblasts, dendritic-like cells, adipocytes, and potentially some myocytes) (Amante D H, et al., Hum Gene Ther Methods, 2015, 26(4):134-146), resulting in different recruitment profiles for antigen-presenting cells to the site of immunization. Additional study in this regard is warranted. ID delivery using synthetic DNA has significant advantages for rapid clinical development, is dose sparing with a simple administration procedure, and is associated with high tolerability.

As MERS vaccine candidates progress through preclinical and clinical studies, questions regarding animal models and efficacy endpoints are important to be addressed. In-country human efficacy trials are likely to be challenging due to the low number of yearly cases (<300). Data from animal models, such as this NHP model, therefore have value as a useful tool to bridge with human data coming from expanded phase clinical trials. Understanding the relevance of rigorous installation challenges in NHPs is important as it is unlikely that humans encounter such a high infectious dose from multiple sites. It is possible that this model is a high bar for vaccine sterilization; however, the vaccines tested in this study still exhibited substantial impact and protection from disease, which was more pronounced using the ID route of vaccination.

The reproducibility of the NHP model of MERS-CoV infection and the clear phenotype of disease induced mimicking aspects of human infection suggested that such a model is also likely to be useful with regards to studies of vaccines for SARS-CoV-2, the virus responsible for the COVID-19 disease pandemic that was first identified in China in 2019. Furthermore, questions have been raised by some vaccine studies in SARS and MERS challenge models reporting enhancement of viral pathogenesis in immunized animals compared to non-vaccinated controls in the absence of robust neutralizing antibodies. For example, a study reported on an Ad5-MERS Spike vaccine, in which a mouse model appeared to increase lung pathogenesis following viral challenge (Hashem A M et al., 2019, J. Infect. Dis., 220:1558-1567). Such enhancement of disease has also been reported for an MVA vectored Spike SARS vaccine in an NHP challenge model where immunized animals presented with diffuse alveolar damage after SARS-CoV challenge whereas control immunized animals showed only signs of minor inflammation after SARS-CoV infection (Liu L et al., 2019, JCI Insight, 4).

In the current study, no evidence of adverse lung pathology was observed in any of the dosing groups compared to unimmunized control animals. Furthermore, assessment of a large panel of blood cytokines post challenge showed significant decreases in all such inflammatory mediators, and were consistently observed across the animals in this challenge, suggesting that the vaccines have a benefit in prevention of virally induced destructive inflammation.

In summary, the results described herein demonstrated that DNA vaccines, even administered in a 2 dose ID regimen, regimen can have positive impact in an important NHP challenge model protecting against symptoms and pathology. ID delivery of a synthetic DNA vaccine encoding a full-length MERS-CoV Spike provided dose-sparing protection against MERS-CoV in a macaque challenge model with no evidence of enhanced lung pathology, and limited virally induced systemic inflammation. In addition, the vaccine induced antibody and cellular immune responses, both of which can contribute to protection and clearance of virally infected cells, limiting pathogenesis and reducing viral loads in MERS-infected patients (Zhao J et al., 2017, Sci. Immunol., 2).

Additional studies and comparison of immunogenicity data from human trials are informative for MERS-CoV as well as for other emerging CoV infections. Data from the MERS DNA vaccine are useful for bridging safety and toxicology data with other emerging coronaviruses and to facilitate evaluation of potential vaccine candidates as they progress through the development pathway.

Overall, emerging coronaviruses from zoonotic reservoirs including severe acute respiratory syndrome coronavirus (SARS-CoV), MERS-CoV, and SARS-CoV-2 have been associated with human-to-human transmission and significant morbidity and mortality. The present example studied both ID and IM two-dose delivery regimens of an advanced synthetic DNA vaccine candidate encoding a full-length MERS-CoV Spike (S) protein, which induced potent binding and neutralizing antibodies and cellular immunity in rhesus macaques. In a MERS-CoV challenge, all immunized rhesus macaques exhibited reduced clinical symptoms, lowered viral lung load, and decreased severity of pathological signs of disease compared to controls. ID vaccination was dose sparing and highly effective in this model at protecting animals from disease and lowering viral loads. The data supported the further study of this vaccine for preventing MERS-CoV infection and transmission and investigation of such vaccines and simplified delivery routes against emerging coronaviruses.

The materials and methods used in the present example are now described:

### Ethics and Biosafety

All animal experiments were approved by the Institutional Animal Care and Use Committee at BIOQUAL (Rockville, Maryland) and at Rocky Mountain Laboratories, NIH and were carried out by certified staff in Association for Assessment and Accreditation of Laboratory Animal Care (AAALAC) International accredited facilities, according to the institution's guidelines for animal use, and followed the guidelines and basic principles in the United States Public Health Service Policy on Humane Care and Use of Laboratory Animals (available from grants.nih.gov/grants/olaw/references/PHSPolicyLabAnimals.pdf), and the Guide for the Care and Use of Laboratory Animals (available from grants.nih.gov/grants/olaw/Guide-for-the-Care-and-use-of-laboratory-animals.pdf).

The Institutional Biosafety Committee (IBC) approved work with infectious MERS-CoV under BSL3 conditions. Sample inactivation was performed according to IBC-approved standard operating procedures for removal of specimens from high containment.

### Study Design

Groups of 6 rhesus macaques were vaccinated twice 4 weeks apart intramuscularly (1 mg—1 site) or intradermally with various doses (2 mg-1 mg in 2 sites; 1 mg—1 site; 0.2 mg-0.1 mg in 2 sites) of a synthetic DNA vaccine encoding a full-length MERS-CoV Spike antigen with electroporation (EP)(Muthumani K et al., 2015, Sci. Transl. Med., 7:301ra132). A subset of animals (IM n=4, ID-high n=4, ID-low n=4, control n=6) were transported from BIOQUAL, Inc. to RML approximately 2 weeks before live virus challenge. Humoral responses were similar for all selected animals and selection was based on their cellular responses post-immunization. Macaques that trended towards higher antibody and T cell levels were selected for challenge, although levels were not significantly different from animals that were not selected. Open symbols in FIG. 1C-E indicate animals not selected for challenge. Animals were randomly assigned study numbers before arrival at RML and all RML personnel were completely blinded to group assignments.

Rhesus macaques were inoculated with 7×10⁶ TCID₅₀ of MERS-CoV EMC/2012, by combination of intratracheal, intranasal, oral, and ocular routes (Munster V J et al., 2013, N. Engl. J. Med., 368:1560-1562). After challenge, the animals were observed twice daily for clinical signs of disease and scored using a previously described clinical scoring system (the same person, blinded to group assignments, scored the animals throughout the entire study) (Brining D L et al., 2010, Comp. Med., 60:389-395).

On 0, 1, 3, 5, and 6 days post inoculation (dpi), clinical exams were performed on anaesthetized animals by board-certified clinical veterinarians. Blood was collected for hematology, serum chemistry, and serological analysis. Ventral-dorsal and lateral radiographs were collected. On 6 dpi all animals were euthanized, and necropsy was performed on all animals by a board-certified veterinary pathologist. Conjunctiva, nasal mucosa, mandibular lymph nodes, tonsils, pharynx, trachea, right and left bronchus, samples from all lung lobes, mediastinal lymph nodes, liver, spleen, kidney and urinary bladder were collected for virological analysis; whole lungs were collected for histopathological analysis.

### Challenge Virus

MERS-CoV EMC/2012 (Vero passage 6) was obtained from the Department of Viroscience, Erasmus Medical Center, Rotterdam, The Netherlands and propagated once in VeroE6 cells in DMEM (Sigma) supplemented with 2% fetal calf serum (Logan), 1 mM L-glutamine (Lonza), 50 U/ml penicillin and 50 μg/ml streptomycin (Gibco) (virus isolation medium). For inoculation of rhesus macaques, virus stock was diluted to the desired titer in DMEM.

### Hematology and Clinical Chemistries

The total white blood cell count, lymphocyte, neutrophil, platelet, reticulocyte, and red blood cell counts, hemoglobin, and hematocrit values were determined from EDTA blood with the IDEXX ProCyte DX analyzer (IDEXX Laboratories). Serum biochemistry (albumin, AST, ALT, GGT, BUN, creatinine) was analyzed using the Piccolo Xpress Chemistry Analyzer and Piccolo General Chemistry 13 Panel discs (Abaxis).

### PBMC Isolation

Whole blood was collected from each NHP into sodium citrate cell preparation tubes (CPT, BD Biosciences) containing an anticoagulant and a gel barrier. Prior to same-day shipment and following collection, the tubes were spun to separate and concentrate PBMCs as per manufacturer's instructions. Red blood cells and neutrophils pellet at the bottom of the tubes and are held in place by the gel barrier. Plasma and lymphocytes remain above the gel barrier. Each CPT can hold ˜8 mL of blood and is shipped at room temperature. The spun CPT tubes were processed for PBMC isolation. After red blood cell lysis with ammonium-chloride-potassium (ACK) buffer, viable cells were counted using ThermoFisher Countess™ Automated Cell Counter and resuspended in complete culture medium media (RPMI 1640 supplemented with 10% FBS, 1% Penicillin/Streptomycin) (herein referred to as R10). After removing cells for IFN-γ ELISpot and ICS assays, the remaining PBMCs were frozen in freezing media (10% DMSO, 10% RPMI, 80% FBS) in cryovials and stored long term in liquid nitrogen.

### Enzyme-Linked Immunospot (ELISPOT)

To assess the cellular IFN-γ responses to vaccinations, Monkey interferon (IFN)-γ enzyme-linked immunospot (ELISPOT) assays were performed using a Mabtech IFN-γ ELISpotPRO (ALP) kit (Cat #3421M-2APW-10, Mabtech, Sweden) following manufacturer's description. Briefly, 96-well plates, same day as PBMC isolation, were blocked for a minimum 2 hours with R10 and then 200,000 PBMCs from study animals were added to each well and incubated at 37° C. in 5% CO2 in the presence of media with DMSO (negative control), or cell stimulation cocktail (PMA/Ionomycin, eBioscience) (positive control for monkey, 5000 cells/well plated), or media with peptide pools consisting 15-mers overlapping by 9 amino acids and spanning the length of MERS Spike protein (GenScript, custom made) or recombinant Spike protein (SinoBiological). After 18-20 hours, the plates were washed and spots were developed, according to the manufacturer's protocol. Antigen-specific responses were determined by subtracting the number of spots in the DMSO containing wells from the wells containing peptides or protein stimulation.

### Enzyme-Linked Immunosorbent Assay (ELISA)

ELISA was performed to determine the antigen-specific antibody response in sera. 96-well ELISA plates (Nunc, 44-2404-21) were coated with 1 μg/ml recombinant MERS Spike, 51 protein, S2 protein, or RBD proteins (SinoBiological) in DPBS overnight at 4° C. Plates were then washed with 4×PBS+0.05% Tween 20 and blocked with 5% Skim Milk in PBS+0.05% Tween 20 for 90 minutes at 37° C. After blocking buffer incubation, plates were washed and serially diluted Rhesus macaque sera was added with dilution buffer (5% skim milk in PBS-Tween20) and incubated for 1 hour at 37° C. Plates were washed and 1:10,000 dilution of secondary antibody HRP conjugate was added and incubated for 1 hour at 37° C. Plates were washed and one-step TMB (Sigma) was applied to the plates, and the reaction was stopped with 2N sulfuric acid. Plates were then read for absorbance at 450 nm within 30 minutes using a Biotek Synergy 2 plate reader. Sera from 24 unvaccinated rhesus macaques was utilized to determine the background cut-off for calculating endpoint titers for each target protein. Sera samples were scored as positive for binding antibodies if they were 3 standard deviations above the average of the unvaccinated animals.

### Virus Neutralization Assay

Two-fold serial dilutions of heat-inactivated (30 minutes, 56° C.) rhesus macaque sera were prepared in DMEM containing 2% fetal calf serum, 1 mM L-glutamine, 50 U/ml penicillin and 50 μg/ml streptomycin, after which 100 TCID₅₀ of HCoV-EMC/2012 virus was added. After 1 hr incubation at 37° C., this mix was added to VeroE6 cells. At 5 dpi, wells were scored for cytopathic effect. The virus neutralization titer was expressed as the reciprocal value of the highest dilution of the serum that still inhibited HCoV-EMC/2012 virus replication.

### Quantitative RT-PCR

Tissues (30 mg) were homogenized in RLT buffer and RNA was extracted using the RNeasy kit (Qiagen) according to the manufacturer's instructions. For detection of viral RNA, 5 μl RNA was used in a one-step real-time RT-PCR upE assay (Corman et al., 2012) using the Rotor-Gene probe kit (Qiagen) according to instructions of the manufacturer. In each run, standard dilutions with known copy numbers of a T7 in vitro-transcribed RNA standard were run in parallel, to calculate the copy number of RNA present in the samples.

### Radiographs

Ventrodorsal and lateral (right and left) radiographs were obtained using a mobile digital radiography unit with a flat-panel digital detector (Sound Technologies tru/DR, Carlsbad, CA) and portable X-ray generator (model PXP-HF, Poskom, Korea). Radiographs were interpreted by two board-certified clinical veterinarians.

### Histopathology

Histopathology and immunohistochemistry were performed on macaque lungs. Tissues were placed in cassettes and fixed in 10% neutral buffered formalin for 7 days. Tissues were subsequently processed with a Sakura VIP-5 Tissue Tek, on a 12-hour automated schedule, using a graded series of ethanol, xylene, and ParaPlast Extra. Embedded tissues were sectioned at 5 μm and dried overnight at 42° C. prior to staining. Tissue sections were stained with hematoxylin and eosin (HE). Specific anti-CoV immunoreactivity was detected using an in-house polyclonal rabbit antibody against MERS-CoV EMC/2012 at a 1:1000 dilution. The tissues were then processed for immunohistochemistry using the Discovery XT automated processor (Ventana Medical Systems) with a DAPMap (Ventana Medical Systems) kit.

### Serum Cytokine and Chemokine Analysis

Serum samples for analysis of cytokine/chemokine levels were inactivated with γ-radiation (5 MRad) according to standard operating procedures. Concentrations of granulocyte colony-stimulating factor, granulocyte-macrophage colony-stimulating factor, interferon (IFN)-γ, interleukin (IL)-1β, IL-1 receptor antagonist, IL-2, IL-4, IL-5, IL-6, IL-8, IL-10, IL-12/23 (p40), IL-13, IL-15, IL-17, MCP-1 and macrophage inflammatory protein (MIP)-1α, MIP-1β, soluble CD40-ligand (sCD40L), transforming growth factor-α, tumor necrosis factor (TNF)-α, vascular endothelial growth factor (VEGF) and IL-18 were measured on a Bio-Plex 200 instrument (Bio-Rad) using the Non-Human Primate Cytokine MILLIPLEX map 23-plex kit (Millipore) according to the manufacturer's instructions.

### Statistical Analyses

GraphPad Prism 7.02/8.0 was used to analyze and plot the data. Data are presented as a range of minimum to maximum value, with all data points shown. Where appropriate, the statistical difference between immunization groups at each time point was assessed using parametric t-test (2-tailed) or nonparametric Mann-Whitney test, adjusted for multiple comparisons using a Bonferroni correction. Adjusted p<0.05 was defined as significant.

It is understood that the foregoing detailed description and accompanying examples are merely illustrative and are not to be taken as limitations upon the scope of the present invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the present invention, may be made without departing from the spirit and scope thereof

1.-53. (canceled)

54. A method of inducing an immune response against a Middle East Respiratory syndrome (MERS) coronavirus (MERS-COV) in a subject in need thereof, the method comprising administering an immunogenic composition intradermally or intramuscularly to the subject, wherein the immunogenic composition comprises a nucleic acid molecule comprising a nucleotide sequence selected from the group consisting of:
a) an immunogenic fragment of SEQ ID NO:1, wherein the fragment encodes a sequence having 100% identity to consecutive amino acids over at least 60% of the full length of SEQ ID NO:2 ;
b) an immunogenic fragment of SEQ ID NO:3, wherein the fragment encodes a sequence having 100% identity to consecutive amino acids over at least 60% of the full length of SEQ ID NO:4;
c) a nucleotide sequence having at least 90% identity over an entire length of the nucleic acid sequence set forth in SEQ ID NO:1;
d) a nucleotide sequence having at least 90% identity over an entire length of the nucleic acid sequence set forth in SEQ ID NO:3;
e an immunogenic fragment of SEQ ID NO:1, wherein the fragment comprises a sequence having 100% identity to consecutive bases over at least 60% of the full length of SEQ ID NO: 1; and
f an immunogenic fragment of SEQ ID NO:3, wherein the fragment comprises a sequence having 100% identity to consecutive bases over at least 60% of the full length of SEQ ID NO:3.

55. The method of claim 54, wherein the method of administering is intradermal administration.

56. The method of claim 55, wherein the method of administering further comprises an electroporation step.

57. The method of claim 54, wherein the composition is administered twice.

58. The method of claim 57, wherein the second administration of the immunogenic composition is given at least 7 days after the first administration.

59. The method of claim 54, wherein the nucleic acid molecule comprises an expression vector.

60. The method of claim 54, wherein the immunogenic composition further comprises a pharmaceutically acceptable excipient, an adjuvant, or a combination thereof.

61. The method of claim 54, wherein the immune response is protective or therapeutic.

62. A method of inducing an immune response against a Middle East Respiratory Syndrome coronavirus (MERS-COV) in a subject in need thereof, the method comprising administering an immunogenic composition intradermally or intramuscularly to the subject, wherein the immunogenic composition comprises an antigen comprising an amino acid sequence selected from the group consisting of:
a) an amino acid sequence as set forth in SEQ ID NO: 2;
b) an amino acid sequence as set forth in SEQ ID NO: 4;
c) a fragment of SEQ ID NO: 2 lacking the IgE leader sequence as set forth by SEQ ID NO: 6; and
d) a fragment of SEQ ID NO: 4 lacking the IgE leader sequence as set forth by SEQ ID NO: 6.

63. The method of claim 62, wherein the method of administering is intradermal administration.

64. The method of claim 62, wherein the method of administering further comprises an electroporation step.

65. The method of claim 62, wherein the composition is administered twice.

66. The method of claim 62, wherein the second administration of the immunogenic composition is given at least 7 days after the first administration.

67. An intradermal vaccine comprising an immunogenic composition, wherein the immunogenic composition comprises a nucleic acid molecule comprising a nucleotide sequence selected from the group consisting of:
a) an immunogenic fragment of SEQ ID NO:1, wherein the fragment encodes a sequence having 100% identity to consecutive amino acids over at least 60% of the full length of SEQ ID NO:2;
b) an immunogenic fragment of SEQ ID NO:3, wherein the fragment encodes a sequence having 100% identity to consecutive amino acids over at least 60% of the full length of SEQ ID NO:4;
c) a nucleotide sequence having at least 90% identity over an entire length of the nucleic acid sequence set forth in SEQ ID NO:1;
d) a nucleotide sequence having at least 90% identity over an entire length of the nucleic acid sequence set forth in SEQ ID NO:3;
e) an immunogenic fragment of SEQ ID NO:1, wherein the fragment comprises a sequence having 100% identity to consecutive bases over at least 60% of the full length of SEQ ID NO:1; and
f) an immunogenic fragment of SEQ ID NO:3, wherein the fragment comprises a sequence having 100% identity to consecutive bases over at least 60% of the full length of SEQ ID NO:3.

68. The intradermal vaccine of claim 67, wherein the vaccine is formulated for administration as a low-dose formula, wherein the low-dose formula is less than 1 mg.


SEQUENCE LISTING (7 sequences)

SEQ ID NO: 1 - DNA, 4068 bp, Artificial Sequence
  MERS-CoV Consensus Spike Antigen
atggactggacttggattctgttcctggtcgccgccgcaactcgcgtgcatagctacgtggatgtcggcccagactctgtgaagagtgcttgcatcgaggtcgatattcagcagacattctttgacaagacttggcctcgaccaatcgacgtgagcaaagccgacggcatcatctacccccagggaaggacttatagtaacatcaccattacataccagggcctgttcccttatcagggcgaccacggagatatgtacgtgtattccgccggacatgctaccgggaccacaccacagaaactgtttgtggcaaattattctcaggacgtgaagcagttcgccaacgggtttgtggtcagaatcggcgccgctgcaaactccactggcaccgtgatcatttccccctctaccagtgccacaatccggaaaatctaccctgcttttatgctgggcagctccgtgggaaacttctctgatgggaagatgggccgcttctttaatcacaccctggtgctgctgccagacggatgcgggacactgctgagggccttctactgtatcctggagcccagaagcggaaatcactgccctgctgggaactcatacaccagctttgccacttatcatacccctgctacagactgttccgatggcaattataaccggaatgcctccctgaactctttcaaggaatactttaatctgcgcaactgcacattcatgtacacttataatatcaccgaggatgaaattctggagtggttcgggatcacacagactgctcagggcgtgcacctgttttctagtcgctacgtcgatctgtatggcggaaacatgttccagtttgccaccctgccagtgtacgacacaattaagtactatagcatcattccccatagtatccgatcaattcagagcgacaggaaggcttgggccgctttctacgtgtataaactgcagcccctgaccttcctgctggatttttctgtggacggatacatcaggagagccattgattgcgggtttaacgacctgagccagctgcactgttcctatgaatctttcgatgtggagtccggggtgtactctgtctcaagctttgaggctaagccatcagggagcgtggtcgagcaggcagaaggcgtggagtgcgacttcagtcccctgctgtcaggcacaccccctcaggtgtacaatttcaaaagactggtctttactaactgtaattacaacctgaccaagctgctgagtctgttctcagtgaacgactttacctgcagccagatctcccctgcagccattgccagcaattgttattcctctctgatcctggattacttctcctatcccctgtctatgaaaagtgacctgtcagtgagttcagcaggccctatctctcagtttaattacaagcagtccttctctaaccccacttgcctgattctggccaccgtgcctcacaacctgactaccatcacaaagccactgaaatactcctatattaacaagtgcagcagactgctgtccgacgatcggactgaagtgcctcagctggtcaatgccaaccagtactctccatgcgtgagcatcgtcccctcaaccgtgtgggaagacggagattactatcggaagcagctgagccccctggagggcggcggctggctggtggcaagtgggtcaacagtcgccatgactgagcagctgcagatgggcttcggaatcaccgtgcagtacggcaccgatacaaattctgtctgtcctaagctggaatttgctaacgacacaaaaattgcaagtcagctgggcaattgcgtggagtactctctgtatggagtgagtgggagaggcgtcttccagaactgtacagccgtgggcgtccgacagcagaggttcgtgtacgatgcttatcagaacctggtcggctactattccgacgatggaaattactattgcctgcgagcatgcgtgagcgtcccagtgtccgtcatctacgacaaggaaactaaaacccacgcaaccctgttcggctcagtggcctgcgagcatattagctccaccatgagccagtatagcagatccacacggtccatgctgaaacggcgcgactctacatacggacccctgcagactcctgtggggtgcgtgctgggcctggtgaactctagtctgttcgtcgaagattgcaagctgccactgggacagtctctgtgcgcactgccagacacacccagtacactgactccacgcagcgtgcgatccgtcccaggagagatgagactggcaagcatcgccttcaatcaccctattcaggtggatcagctgaactcaagctactttaagctgtcaatcccaacaaacttcagctttggcgtgactcaggagtatatccagacaactattcagaaggtgaccgtcgactgcaaacagtacgtgtgcaatggattccagaaatgcgaacagctgctgcgggagtatgggcagttttgttccaagatcaatcaggcactgcatggcgccaacctgcgccaggacgatagtgtgcgaaacctgttcgcctcagtcaagtcctctcagagttcacctatcattccagggttcggcggcgacttcaacctgaccctgctggaacccgtgagcatcagtaccggcagcaggagcgccagaagcgcaatcgaggatctgctgtttgacaaagtgaccattgccgacccaggatacatgcaggggtatgacgattgcatgcagcagggaccagcatccgctcgcgatctgatctgtgctcagtacgtggcagggtataaggtcctgccacccctgatggacgtgaacatggaagctgcatatactagctccctgctggggagcattgcaggagtgggatggaccgctggactgtctagtttcgccgctatcccatttgctcagagcattttctacaggctgaacggcgtgggaatcactcagcaggtcctgtccgagaatcagaagctgattgccaacaagttcaaccaggccctgggagctatgcagaccgggtttaccacaactaacgaagctttccgcaaagtgcaggacgcagtcaacaataacgcacaggccctgtccaagctggcttctgagctgagtaatacattcggagcaatctccgcctctattggggatatcattcagaggctggacgtgctggagcaggatgcccagatcgaccggctgattaatggacgcctgaccacactgaacgcttttgtggcacagcagctggtccgaagtgaatcagcagccctgtctgcccagctggctaaggacaaagtgaacgagtgcgtcaaggctcagtcaaaacggagcggcttttgtgggcagggcacccacatcgtgagcttcgtggtcaatgcacctaacggcctgtactttatgcacgtgggatactatccaagcaaccatatcgaggtggtctccgcttatggcctgtgcgatgctgcaaatcctacaaactgtattgcaccagtgaacggatacttcatcaaaactaacaacaccaggattgtggacgaatggtcatacactggctcaagcttttatgcacccgagcctatcacctccctgaacacaaagtacgtggccccacatgtcacctatcagaatatctccacaaacctgcctccacccctgctgggcaattctaccggaattgacttccaggatgaactggacgagttctttaagaatgtgagcacatccatccccaactttggaagcctgactcagattaacactaccctgctggatctgacctacgagatgctgagtctgcagcaggtggtcaaggccctgaatgaatcatacatcgacctgaaagagctggggaattatacatactataacaagtggccctggtacatctggctggggttcattgcaggactggtggctctggcactgtgcgtcttctttatcctgtgctgtactggatgcgggaccaactgtatgggcaagctgaaatgtaatcggtgttgtgatcgctacgaagaatacgacctggagccccataaagtgcatgtccactaatga

SEQ ID NO: 2 - protein, 1354 aa, Artificial Sequence
  MERS-CoV Consensus Spike Antigen
MDWTWILFLVAAATRVHSYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPHVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 3 - DNA, 4074 bp, Artificial Sequence
  MERS-CoV Consensus Spike Antigen lacking Cytoplasmic Domain
atggactggacttggattctgttcctggtcgccgccgcaactcgcgtgcatagctacgtggatgtcggcccagactctgtgaagagtgcttgcatcgaggtcgatattcagcagacattctttgacaagacttggcctcgaccaatcgacgtgagcaaagccgacggcatcatctacccccagggaaggacttatagtaacatcaccattacataccagggcctgttcccttatcagggcgaccacggagatatgtacgtgtattccgccggacatgctaccgggaccacaccacagaaactgtttgtggcaaattattctcaggacgtgaagcagttcgccaacgggtttgtggtcagaatcggcgccgctgcaaactccactggcaccgtgatcatttccccctctaccagtgccacaatccggaaaatctaccctgcttttatgctgggcagctccgtgggaaacttctctgatgggaagatgggccgcttctttaatcacaccctggtgctgctgccagacggatgcgggacactgctgagggccttctactgtatcctggagcccagaagcggaaatcactgccctgctgggaactcatacaccagctttgccacttatcatacccctgctacagactgttccgatggcaattataaccggaatgcctccctgaactctttcaaggaatactttaatctgcgcaactgcacattcatgtacacttataatatcaccgaggatgaaattctggagtggttcgggatcacacagactgctcagggcgtgcacctgttttctagtcgctacgtcgatctgtatggcggaaacatgttccagtttgccaccctgccagtgtacgacacaattaagtactatagcatcattccccatagtatccgatcaattcagagcgacaggaaggcttgggccgctttctacgtgtataaactgcagcccctgaccttcctgctggatttttctgtggacggatacatcaggagagccattgattgcgggtttaacgacctgagccagctgcactgttcctatgaatctttcgatgtggagtccggggtgtactctgtctcaagctttgaggctaagccatcagggagcgtggtcgagcaggcagaaggcgtggagtgcgacttcagtcccctgctgtcaggcacaccccctcaggtgtacaatttcaaaagactggtctttactaactgtaattacaacctgaccaagctgctgagtctgttctcagtgaacgactttacctgcagccagatctcccctgcagccattgccagcaattgttattcctctctgatcctggattacttctcctatcccctgtctatgaaaagtgacctgtcagtgagttcagcaggccctatctctcagtttaattacaagcagtccttctctaaccccacttgcctgattctggccaccgtgcctcacaacctgactaccatcacaaagccactgaaatactcctatattaacaagtgcagcagactgctgtccgacgatcggactgaagtgcctcagctggtcaatgccaaccagtactctccatgcgtgagcatcgtcccctcaaccgtgtgggaagacggagattactatcggaagcagctgagccccctggagggcggcggctggctggtggcaagtgggtcaacagtcgccatgactgagcagctgcagatgggcttcggaatcaccgtgcagtacggcaccgatacaaattctgtctgtcctaagctggaatttgctaacgacacaaaaattgcaagtcagctgggcaattgcgtggagtactctctgtatggagtgagtgggagaggcgtcttccagaactgtacagccgtgggcgtccgacagcagaggttcgtgtacgatgcttatcagaacctggtcggctactattccgacgatggaaattactattgcctgcgagcatgcgtgagcgtcccagtgtccgtcatctacgacaaggaaactaaaacccacgcaaccctgttcggctcagtggcctgcgagcatattagctccaccatgagccagtatagcagatccacacggtccatgctgaaacggcgcgactctacatacggacccctgcagactcctgtggggtgcgtgctgggcctggtgaactctagtctgttcgtcgaagattgcaagctgccactgggacagtctctgtgcgcactgccagacacacccagtacactgactccacgcagcgtgcgatccgtcccaggagagatgagactggcaagcatcgccttcaatcaccctattcaggtggatcagctgaactcaagctactttaagctgtcaatcccaacaaacttcagctttggcgtgactcaggagtatatccagacaactattcagaaggtgaccgtcgactgcaaacagtacgtgtgcaatggattccagaaatgcgaacagctgctgcgggagtatgggcagttttgttccaagatcaatcaggcactgcatggcgccaacctgcgccaggacgatagtgtgcgaaacctgttcgcctcagtcaagtcctctcagagttcacctatcattccagggttcggcggcgacttcaacctgaccctgctggaacccgtgagcatcagtaccggcagcaggagcgccagaagcgcaatcgaggatctgctgtttgacaaagtgaccattgccgacccaggatacatgcaggggtatgacgattgcatgcagcagggaccagcatccgctcgcgatctgatctgtgctcagtacgtggcagggtataaggtcctgccacccctgatggacgtgaacatggaagctgcatatactagctccctgctggggagcattgcaggagtgggatggaccgctggactgtctagtttcgccgctatcccatttgctcagagcattttctacaggctgaacggcgtgggaatcactcagcaggtcctgtccgagaatcagaagctgattgccaacaagttcaaccaggccctgggagctatgcagaccgggtttaccacaactaacgaagctttccgcaaagtgcaggacgcagtcaacaataacgcacaggccctgtccaagctggcttctgagctgagtaatacattcggagcaatctccgcctctattggggatatcattcagaggctggacgtgctggagcaggatgcccagatcgaccggctgattaatggacgcctgaccacactgaacgcttttgtggcacagcagctggtccgaagtgaatcagcagccctgtctgcccagctggctaaggacaaagtgaacgagtgcgtcaaggctcagtcaaaacggagcggcttttgtgggcagggcacccacatcgtgagcttcgtggtcaatgcacctaacggcctgtactttatgcacgtgggatactatccaagcaaccatatcgaggtggtctccgcttatggcctgtgcgatgctgcaaatcctacaaactgtattgcaccagtgaacggatacttcatcaaaactaacaacaccaggattgtggacgaatggtcatacactggctcaagcttttatgcacccgagcctatcacctccctgaacacaaagtacgtggccccacatgtcacctatcagaatatctccacaaacctgcctccacccctgctgggcaattctaccggaattgacttccaggatgaactggacgagttctttaagaatgtgagcacatccatccccaactttggaagcctgactcagattaacactaccctgctggatctgacctacgagatgctgagtctgcagcaggtggtcaaggccctgaatgaatcatacatcgacctgaaagagctggggaattatacatactataacaagtggccctggtacatctggctggggttcattgcaggactggtggctctggcactgtgcgtcttctttatctaatgactgtgctgtactggatgcgggaccaactgtatgggcaagctgaaatgtaatcggtgttgtgatcgctacgaagaatacgacctggagccccataaagtgcatgtccactaatga

SEQ ID NO: 4 - protein, 1318 aa, Artificial Sequence
  MERS-CoV Consensus Spike Antigen lacking Cytoplasmic Domain
MDWTWILFLVAAATRVHSYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPHVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFI

SEQ ID NO: 5 - DNA, 54 bp, Artificial Sequence
  IgE leader sequence
atggactggacttggattctgttcctggtcgccgccgcaactcgcgtgcatagc

SEQ ID NO: 6 - protein, 18 aa, Artificial Sequence
  IgE leader sequence
MDWTWILFLVAAATRVHS

SEQ ID NO: 7 - protein, 15 aa, Artificial Sequence
  Synthetic polypeptide
RKAWAAFYVYKLQPL